(19) United States
(12) Patent Application Publication
(10) Pub. No.: US 2010/0047843 A1
(43) Pub. Date: Feb. 25, 2010

(54) COMPOSITIONS AND METHODS FOR ENHANCING THE GROWTH OF MOUSE EMBRYONIC STEM CELLS

(76) Inventor: Tobias D. Raabe, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/520,662

(22) PCT Filed: Dec. 22, 2007
(86) PCT No.: PCT/US07/88756

§ 371 Date: Oct. 30, 2009

Related U.S. Application Data
(60) Provisional application No. 60/871,484, filed on Dec. 22, 2006.

(51) Int. Cl. C12Q 1/02; C12N 5/0735; C07K 2/00

(52) U.S. Cl. 435/29; 435/354; 530/300

(57) ABSTRACT

Compositions and methods are provided which improve the growth rate, self-renewal potential and capacity of germ line transmission of mouse embryonic stem cells.

This application claims priority under 35 U.S.C. §119(e) to U.S. Provisional Patent Application 60/871,484, filed on Dec. 22, 2006. The foregoing application is incorporated by reference herein.

Pursuant to 35 U.S.C. §202(c) it is acknowledged that the U.S. Government has certain rights in the invention described, which was made in part with funds from the National Institutes of Health, Grant Number 1-U01-DA021912-01.

### FIELD OF THE INVENTION

This invention relates to the fields of transgenic animal production and to methods for propagation of embryonic stem cells. More specifically, the invention provides compositions and methods for enhancing growth, viability and frequency of germ line transmission of mouse ES cells.

### BACKGROUND OF THE INVENTION

Several publications and patent documents are cited throughout the specification in order to describe the state of the art to which this invention pertains. Each of these citations is incorporated by reference herein as though set forth in full.

With the advent of gene targeting in mouse embryonic stem (ES) cells and the derivation of mice from mutated ES cells in the mid 1980s, mouse genetics has revolutionized biomedical research. The increase in the number of applications of mouse knock-out technology has been dramatic, generating hundreds of models of human disease for mechanistic analysis and evaluation of novel therapeutic modalities. Today, it is possible to modify the mouse genome at will, from the derivation of single point mutations to engineered chromosomal translocations and mega-base deletions, to the conditional inactivation of genes in only a single specific cell type and at a chosen time point in development. Thus, modern mouse genetics is a powerful and important tool in the study of human disease. Given the value of mutant mice and the completion of the mouse genome and its annotation with cDNAs, it is clear that the next massive effort that could promote mammalian genetics and biomedical research is the establishment of a compendium of freely accessible knockout strains for all mouse genes (1).

The vast majority of mutant mice generated by gene targeting/trapping have been derived in one of the roughly twenty 129 mouse substrains (2). These substrains are not genetically uniform, increasing the complexity of phenotypic analysis, and sometimes obscuring important functions of the inactivated genes (3). In addition, many 129 mice are not suitable for studies involving behavioral testing as they exhibit a high base level of divergent behavioral patterns (4,5).

Ideally, gene targeting/trapping should be performed in a single genetic background that has been proven to be useful for the creation of many different kinds of disease models and analytical modalities. The C57BL/6 (B6) strain is completely inbred and thus genetically uniform and has been widely used as a standardized model for biochemical, histological and behavioral paradigms (4,5,6). The C57BL/6 is also the only mouse strain whose genome has been fully sequenced to date and whose sequence is freely available to the public. An important advantage of the B6 strain for high throughput gene targeting is the fact that large BAC libraries have been end-sequenced and mapped to the genome. This information is also in the public domain. It has been shown a decade ago that the use of isogenic DNA greatly increases the frequency of homologous recombination in gene targeting experiments (7). Thus, it is trivial to obtain the relevant B6 BAC clone containing the gene in question and construct a targeting vector that is isogenic, that is identical in sequence, to the genome of B6 embryonic stem cells for optimal targeting frequency. Thus, the C57BL/6 mouse strain was chosen by the mouse genetics community as the ideal strain for use in the mouse knockout project (KOMP (4)). However, a major hurdle towards the use of the B6 ES cells in high throughput gene targeting/trapping is their reduced frequency of germ line transmission compared to 129 ES cells (6). Thus, methods and compositions which increase the frequency of germ line transmission in this mouse strain are highly desirable.

### SUMMARY OF THE INVENTION

In accordance with the present invention, compositions and methods which result in enhanced growth, self-renewal, frequency of germ line transmission and viability of mouse embryonic stem cells are provided. An exemplary method for enhancing the growth of mouse embryonic stem cells on non-confluent feeder layers comprises supplementing the culture medium with conditioned ES/MEF culture medium. Preferably, the embryonic stem cells are B6 cells and the ES/MEF culture medium is obtained from cultured mouse 129 ES cells. Optionally, the supplemented growth medium further comprises BMP 3/4 and/or GDF6. The supplemented growth medium may also contain at least one growth factor selected from the group consisting of BMP3/4, GDF6, bFGF and TGF beta.

Another embodiment of the invention is directed to a method for culturing mouse ES cells in the absence of feeder layers which comprises seeding the cells onto a matrix and supplementing the growth medium with conditioned ES/MEF culture medium.

Additionally, the invention provides methods for identifying at least one growth factor or cytokine present in cultured medium and isolation of the same.

In yet another embodiment, a method for identifying factors which promote B6 cell survival in cultures lacking feeder cells is provided. An exemplary embodiment entails providing cultures of B6 cells, which are incubated in the presence of i) unconditioned (normal) medium (UCM); ii) MEF conditioned medium (MEF); or iii) 129+MEF conditioned medium; passaging the cells at least five times in the absence of MEF; determining cell viability and proliferation in i), ii) and iii); and purifying at least one factor from the medium of ii) thereby isolating said factor(s) which increase B6 cell survival. Growth factors identified by the method described above are also encompassed by the invention. The method can further comprising isolating and purifying at least one growth factor from the media of iii). Growth factors so identified are also within the scope of the invention.

### BRIEF DESCRIPTION OF THE DRAWINGS

FIG. 1. 129 ES cell-conditioned medium, BMP4 and GDF-6 strongly enhance B6 ES cell growth characteristics and make them less MEF-dependent. Phase contrast images taken from the wells without MEF's of the experiment described in Table 2. 2×10⁵ B6 ES cells were added to wells of a gelatinized 24 well tissue culture plate completely lacking MEF's. All residual MEF's seen in these photographs stem directly from the B6 ES cell suspension used for inoculation. I added normal medium or 129-conditioned medium and/or 20 ng/ml recombinant BMP4 (R&D) plus 300 ng/ml recombinant GDF-6 (R&D) per well, as indicated. 26 h after seeding the B6 ES cells wells were photographed. Note that only few MEF's are present and that many colonies are attached directly to the plastic of the 24 well plate.

FIG. 2. Matrigel allows feederless growth of B6 ES cells with undifferentiated morphology. B6 ES cells were grown for three passages without feeder cells on gelatin in the presence of 129-conditioned medium. They were then trypsinized as usual and equal amounts were added to either gelatin (A) or matrigel (B) coated plates together with 129 conditioned medium. Coating of plates with matrigel was done according to manufacturers recommendations. After 24 h of culture pictures were taken. Most colonies consist of 2-4 ES cells.

FIG. 3: The ‘B6 survival test’ shows that MEF alone or MEF+129 ES cells excrete ‘B6 survival factors’ that are necessary for growth of B6 ES cells in the absence of MEF. The ‘B6 survival test’ was done as follows: 3 million B6 ES cells were added to one 12 well of a 12 well plate and passaged 5 times in the absence of MEF. Passaging was done by normal trypsinization, and the cells were split at a ratio of about 1:5. Cells were either grown with unconditioned (normal) medium (UCM), MEF conditioned medium (MEF) or 129+MEF conditioned medium also called 129 conditioned medium (R1+MEF; R1 ES cells are one type of 129 ES cells). Black bars are error bars. The blue bars represent averages of 12 independent experiments that were done on 3 different dates with 3 different preparations of each type of medium. The scale indicates millions of cells per 12 well of a 12 well culture dish.

FIG. 4: ‘B6 survival factors’ can be frozen without loss of activity and can be excreted into serum free medium. B6 ES cells were passaged 5 times in the absence of MEF as in FIG. 3 and survival was measured by counting the cells. Averages of 3 independent experiments are given. B6 ES cells were either grown in normal unconditioned medium (UC Normal), MEF conditioned medium that was frozen once (Frozen MEF normal), serum free unconditioned medium (UC Serum Free) or serum free MEF conditioned medium that was frozen once (Frozen MEF Serum Free). The serum free MEF conditioned medium was made by growing confluent MEF for 2 days in normal medium and then washing two times with PBS and growing for a further 24 h in the presence of Optimem serum free medium (Gibco-Invitrogen). At the end of the incubation period it was filtered through a 0.2 micron filter and frozen immediately.

### DETAILED DESCRIPTION OF THE INVENTION

In accordance with the present invention, it has been discovered that the reduced rate of growth and increased rate of spontaneous differentiation observed in B6 ES cells compared to 129 ES cells (6) does not appear to be attributable to an inherently lower capacity for growth or inherent higher capacity to differentiate in B6 ES cells but rather is due to the fact that the range of growth conditions supporting self renewal is considerably narrower for B6 than 129 ES cell lines. This is particularly true with respect to the density of mouse embryonic fibroblast feeder layers: at high feeder density B6 cells grow slowly, and at low feeder density they differentiate into fibroblast-type cells with concomitant loss of pluripotency. Accordingly, compositions and methods have been developed which markedly improve germ line transmission rates for B6 cells.

The improved cell culture conditions described herein facilitate the use of B6 ES cells in high throughput settings. Not only all B6 ES cell lines, present and future, but potentially all ES cells in general that grow slowly and have a higher propensity to differentiate under normal growth conditions could exhibit improved growth and self renewal characteristics, including higher rates of germ line transmission, if grown under the new culture conditions.

The following definitions are provided to facilitate an understanding of the invention.

“Conditioned ES/MEF culture medium” refers to growth factor/cytokine containing media obtained from vessels where ES and/or MEF cells have been cultured. Preferably, the medium is obtained from cultures that have been grown for at least 24 h after splitting or at least about 5 h after the first medium change after splitting.

An exemplary “normal culture medium” can refer to media containing the following standard components:

| | | |
|---|---|---|
| DMEM | 400 | ml |
| FCS (ES cell qualified) −20° C. | 80 | ml |
| L-glu 100X (−20° C.) | 5 | ml |
| NEAA 100X (4° C.) | 5 | ml |
| HEPES 1M (4° C.) | 5 | ml |
| Pen/Strep (antibiotic) 100X (4° C.) | 5 | ml |
| LIF (10⁷ u/ml) (4° C.) | 50 | μl |
| β-Mercaptoethanol 55 mM (4° C.) | 500 | μl |
| (Tissue Culture Grade) | | |

However the skilled person appreciates that many standard media are available for cell culture and are commercially available from Invitrogen and Gibco, BRL.

The term “embryonic stem cell” as used herein can refer to pluripotent cells isolated from an embryo that are maintained in in vitro cell culture. Such cells are rapidly dividing cultured cells isolated from cultured embryos which retain in culture the ability to give rise, in vivo, to all the cell types which comprise the adult animal, including the germ cells. Embryonic stem cells may be cultured with or without feeder cells. Embryonic stem cells can be established from embryonic cells isolated from embryos at any stage of development, including blastocyst stage embryos and pre-blastocyst stage embryos. Embryonic stem cells may have a rounded cell morphology and may grow in rounded cell clumps on feeder layers. Embryonic stem cells are well known to a person of ordinary skill in the art. See, e.g., WO 97/37009, entitled “Cultured Inner Cell Mass Cell-Lines Derived from Ungulate Embryos,” Stice and Golueke, published Oct. 9, 1997, and Yang & Anderson, 1992, Theriogenology 38: 315-335. See, e.g., Piedrahita et al. (1998) Biol. Reprod. 58: 1321-1329; Wianny et al. (1997) Biol. Reprod. 57: 756-764; Moore & Piedrahita (1997) In Vitro Cell Biol. Anim. 33: 62-71; Moore, & Piedrahita, (1996) Mol. Reprod. Dev. 45: 139-144; Wheeler (1994) Reprod. Fert. Dev. 6: 563-568; Hochereau-de Reviers & Perreau, Reprod. Nutr. Dev. 33: 475-493; Strojek et al., (1990) Theriogenology 33: 901-903; Piedrahita et al., (1990) Theriogenology 34: 879-901; and Evans et al., (1990) Theriogenology 33: 125-129.

The term “differentiated cell” as used herein can refer to a precursor cell that has developed from an unspecialized phenotype to a specialized phenotype. For example, embryonic cells can differentiate into an epithelial cell lining the intestine.

The term “undifferentiated cell” as used herein can refer to a precursor cell that has an unspecialized phenotype and is capable of differentiating. An example of an undifferentiated cell is a stem cell.

“Multipotent” implies that a cell is capable, through its progeny, of giving rise to several different cell types found in the adult animal.

“Pluripotent” implies that a cell is capable, through its progeny, of giving rise to all the cell types which comprise the adult animal including the germ cells. Both embryonic stem and embryonic germ cells are pluripotent cells under this definition.

“Self renewal” is a capability of ES cells that allows them to reproduce without changing their properties, which means without differentiation. “Self renewal” allows ES cells at least theoretically to multiply indefinitely without any change of their characteristics, including in their potential for germ line transmission.

“Germ line transmission” is the term used for the ability of ES cells to form a functional gonad after being combined with a host early embryo (usually blastocyst) and implanted into a foster mother. In the uterus of the foster mother these genetically chimeric blastocysts can grow into pups that have their gonads (usually a testes as most ES cell lines used are derived from male embryos) derived from the original ES cells. This testes in turn produces functional sperm (and the ovary produces functional eggs) that, when used to inseminate a recipient female animal, produces viable offspring.

“Frequency of germ line transmission” refers to the fact that most ES cell lines that are combined with host embryo tissue (usually blastocyst stage embryo's) form a functional testes only in a certain fraction of the resulting chimeric animals. For example, a high frequency of germline transmission would indicate that more than 50% of chimeric animals derived from the same ES cell line would contain a functional testes. A low frequency would be if less than 30% of the chimeric animals derived from the same ES cell line contain a functional testes.

A “reconstructed embryo” is an embryo made by the fusion of an enucleated oocyte with a donor somatic or embryonic stem (ES) or embryonic germ (EG) cell; alternatively, the donor cell nucleus can be isolated and injected into the oocyte. In yet another approach chromatin or nuclear DNA may be injected into the oocyte to create the reconstructed embryo.

The term “transgenic” animal or cell refers to animals or cells whose genome has been subject to technical intervention including the addition, removal, or modification of genetic information. The term “chimeric” refers an entity such as an individual, organ, cell, nucleic acid or part thereof consisting of regions derived from entities of diverse genetic constitution.

A “zygote” refers to a fertilized one-cell embryo.

The term “totipotent” as used herein can refer to a cell that gives rise to a live born animal. The term “totipotent” can also refer to a cell that gives rise to all of the cells in a particular animal. A totipotent cell can give rise to all of the cells of an animal when it is utilized in a procedure for developing an embryo from one or more nuclear transfer steps. Totipotent cells may also be used to generate incomplete animals such as those useful for organ harvesting, e.g., having genetic modifications to eliminate growth of an organ or appendage by manipulation of a homeotic gene. Additionally, genetic modification rendering oocytes, such as those derived from ES cells, incapable of development in utero would ensure that human derived ES cells could not be used to derive human oocytes for reproduction and only for applications such as therapeutic cloning.

The term “cultured” as used herein in reference to cells can refer to one or more cells that are undergoing cell division or not undergoing cell division in an in vitro environment. An in vitro environment can be any medium known in the art that is suitable for maintaining cells in vitro, such as suitable liquid media or agar, for example. Specific examples of suitable in vitro environments for cell cultures are described in Culture of Animal Cells: a manual of basic techniques (3.sup.rd edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; and Animal Cells: culture and media, 1994, D. C. Darling, S. J. Morgan John Wiley and Sons, Ltd.

The term “cell line” as used herein can refer to cultured cells that can be passaged at least one time without terminating. The invention relates to cell lines that can be passaged indefinitely. Cell passaging is defined hereafter.

The term “suspension” as used herein can refer to cell culture conditions in which cells are not attached to a solid support. Cells proliferating in suspension can be stirred while proliferating using apparatus well known to those skilled in the art.

The term “monolayer” as used herein can refer to cells that are attached to a solid support while proliferating in suitable culture conditions. A small portion of cells proliferating in a monolayer under suitable growth conditions may be attached to cells in the monolayer but not to the solid support. Preferably less than 15% of these cells are not attached to the solid support, more preferably less than 10% of these cells are not attached to the solid support, and most preferably less than 5% of these cells are not attached to the solid support.

The term “plated” or “plating” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells of the invention can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel which has been supplemented with medium suitable for further cell proliferation.

The term “proliferation” as used herein in reference to cells can refer to a group of cells that can increase in number over a period of time.

The term “permanent” or “immortalized” as used herein in reference to cells can refer to cells that may undergo cell division and double in cell numbers while cultured in an in vitro environment a multiple number of times until the cells terminate. A permanent cell line may double over 10 times before a significant number of cells terminate in culture. Preferably, a permanent cell line may double over 20 times or over 30 times before a significant number of cells terminate in culture. More preferably, a permanent cell line may double over 40 times or 50 times before a significant number of cells terminate in culture. Most preferably, a permanent cell line may double over 60 times before a significant number of cells die in culture.

The term “isolated” as used herein can refer to a cell that is mechanically separated from another group of cells. Examples of a group of cells are a developing cell mass, a cell culture, a cell line, and an animal.

The term “thawing” as used herein can refer to a process of increasing the temperature of a cryopreserved cell, embryo, or portions of animals. Methods of thawing cryopreserved materials such that they are active after a thawing process are well-known to those of ordinary skill in the art.

The terms “transfected” and “transfection” as used herein refer to methods of delivering exogenous DNA into a cell. These methods involve a variety of techniques, such as treating cells with high concentrations of salt, an electric field (“electroporation”), liposomes, polycationic micelles, or detergent, to render a host cell outer membrane or wall permeable to nucleic acid molecules of interest. These specified methods are not limiting and the invention relates to any transformation technique well known to a person of ordinary skill in the art.

The term “antibiotic” as used herein can refer to any molecule that decreases growth rates of a bacterium, yeast, fungi, mold, or other contaminants in a cell culture. Antibiotics are optional components of cell culture media. Examples of antibiotics are well known in the art. See Sigma and DIFCO catalogs.

The term “feeder cells” as used herein can refer to cells that are maintained in culture and are co-cultured with target cells. Target cells can be precursor cells, embryonic stem cells, embryonic germ cells, cultured cells, and totipotent cells, for example. Feeder cells can provide, for example, peptides, polypeptides, electrical signals, organic molecules (e.g., steroids), nucleic acid molecules, growth factors (e.g., bFGF), other factors (e.g., cytokines such as LIF and steel factor), and metabolic nutrients to target cells. Certain cells, such as embryonic germ cells, cultured cells, and totipotent cells may not require feeder cells for healthy growth. Feeder cells preferably grow in a mono-layer.

Feeder cells can be established from multiple cell types. Examples of these cell types are fetal cells, mouse cells, Buffalo rat liver cells, and oviductal cells. These examples are not meant to be limiting. Tissue samples can be broken down to establish a feeder cell line by methods well known in the art (e.g., by using a blender). Feeder cells may originate from the same or different animal species as precursor cells. Feeder cells can be established from ungulate fetal cells, mammalian fetal cells, and murine fetal cells. One or more cell types can be removed from a fetus (e.g., primordial germs cells, cells in the head region, and cells in the body cavity region) and a feeder layer can be established from those cells that have been removed or cells in the remaining dismembered fetus. When an entire fetus is utilized to establish fetal feeder cells, feeder cells (e.g., fibroblast cells) and precursor cells (e.g., primordial germ cells) can arise from the same source (e.g., one fetus).

The term “receptor ligand cocktail” as used herein can refer to a mixture of one or more receptor ligands. A receptor ligand can refer to any molecule that binds to a receptor protein located on the outside or the inside of a cell. Receptor ligands can be selected from molecules of the cytokine family of ligands, bone morphogenic proteins, neurotrophin family of ligands, growth factor family of ligands, and mitogen family of ligands. Examples of receptor/ligand pairs are: epidermal growth factor receptor/epidermal growth factor, insulin receptor/insulin, cAMP-dependent protein kinase/cAMP, growth hormone receptor/growth hormone, and steroid receptor/steroid. It has been shown that certain receptors exhibit cross-reactivity. For example, heterologous receptors, such as insulin-like growth factor receptor 1 (IGFR1) and insulin-like growth factor receptor 2 (IGFR2) can both bind IGF1. When a receptor ligand cocktail comprises a stimulus, the receptor ligand cocktail can be introduced to a precursor cell in a variety of manners known to a person of ordinary skill in the art.

The term “cytokine” as used herein refers to a large family of receptor ligands. The cytokine family of receptor ligands includes such members as leukemia inhibitor factor (LIF); cardiotrophin 1 (CT-1); ciliary neurotrophic factor (CNTF); stem cell factor (SCF), which is also known as Steel factor; oncostatin M (OSM); and any member of the interleukin (IL) family, including IL-6, IL-1, and IL-112. The teachings of the invention do not require the mechanical addition of steel factor (also known as stem cell factor in the art) for the conversion of precursor cells into totipotent cells.

The following examples are provided to illustrate embodiments of the invention. They are not intended to limit the invention in any way.

The materials and methods set forth below facilitate the practice of the present invention.

### Chromosome Counting (Karyotyping)

### Materials:

10 μg/ml Colcemid (Gibco, 15212-012) to arrest the cells at Metaphase
75 mM KCL (hypotonic solution to make the cells swelling).
3:1 Methanol-Acetic Acid (fixative solution) to fix the cell membrane
Hoescht 33258 fluorescent dye 5 ug/ml
Slide (positively charged) and cover slip
Fluorescent Microscope 100× oil immersion objective with digital imaging
irradiated MEF's

### Cell Culture

Seed ES cells on a 10 cm plate with iMEF's (irradiated MEF's)
Split them (1:2) or (1:3) onto the gelatinized 10 cm plates without iMEF
At appropriate density, add 10 ug/ml Colcemid (Gibco) so that the final concentration is 0.1 to 0.2 ug/ml
Incubate at 37° C. for 2 to 4 hours

### Cell Suspension and Fixation

Remove media, wash the plate twice with PBS (gently)
Add freshly thawed 0.05% Trypsin
Optionally trypsininize the 2 plates under 2 different times points.
Stop the trypsin at 3 min on 1 plate and at 6 min on the other after all cells are detached by adding medium.
Transfer suspension to a 15 ml tube
Spin at 1000 rpm for 2 minutes
Remove the supernatant.
Add approximately 0.3 ml of 75 mM KCl solution to the tube and rapidly resuspend the cell pellent with a 200 μl pipette tip which has been cut to widen the end.
Add 5.0 ml of 75 mM KCl (hypotonic solution) and incubate at room temperature for 30 minutes.
Optional: assess spreading of chromosomes after 20, 25 minutes
Add 5 ml of 3:1 (Methanol:Acetic Acid) to fix the cells at least 10 minutes.
Spin and Remove the supernatant and add fresh 3:1 fixative solution. (repeat this step at least once)

### Making Slides and Imaging

Place a slide on paper towel on the floor
Add 3:1 fixative solution drop wise from height
Tilt to drain off most of the fixative
Drop the fixed cells on to the slide from about 2-3 feet above
Observe under phase contrast microscope to check for clear metaphases
Add Hoechst stain in water or mounting medium (2 ng/ml) and cover with cover slip-Seal with nail polish and take digital pictures with 100× Oil immersion objective mounted on fluorescent microscope
process pictures using adobe photoshop, print pictures and count the chromosomes

### Irradiation Protocol for Mouse Embryonic Fibroblasts (MEF's)

### Irradiation

Primary mouse embryonic fibroblasts (MEFs) are prepared following standard protocols

Grow one vial of Chemicon MEF's (non treated) in MEF medium on a 15 cm gelatinized tissue culture plate. After about three days split 1:3 onto 3 15 cm plates. After two more 1:3 splits 27 15 cm plates of confluent MEF's are obtained.

Remove all of the medium

Wash twice with PBS (15 ml each)

Add 5 ml of trypsin (0.05% trypsin) to each plate

Monitor the reaction carefully until all cells are detached

Add 5 ml of medium into each plate to stop trypsinization

Transfer the cell suspension into a 50 ml centrifuge tube

Wrap the tube with parafilm.

Irradiate the cells 25 min in the gamma irradiator model

### Freezing

Remove 50 ml tube from the irradiator, centrifuge cells to remove all medium

Add 50 ml 1×MEF freezing mix (10% DMSO, 50% serum in MEF medium) and resuspend the cells

Transfer 1 ml of the well-suspended cells into cryovials.

Place vials in Styrofoam box and move to −80° C.

On the next day, transfer vials from −80° C. into liquid Nitrogen for long-term storage.

Thaw one vial and grow MEFs to test for mycoplasma contamination

Electroporation of ES Cells with DNA Construct

### Preparation of Cells Before Electroporation

Prepare three 15 cm plates of ES-cells with appropriate colony size and density (approximately ⅔ confluent)

Change the media 2-8 hours before the experiment.

### Electroporation Procedure

Remove medium, wash 2 times with PBS

Add 5 ml of Trypsin in each plate and incubate until the cells are detached.

Add 5 ml of medium in each plate to stop the reaction.

Transfer cells into a 50 ml centrifuge tube.

Centrifuge at 1,500 rpm for 5 minutes.

Remove the supernatant and add PBS (with Ca2+ and Mg2+) so that the final volume is about 2 ml.

Use a 200 ul pipette tip and a Pasteur pipette to draw up and down 5-10 times slowly to resuspend the cells.

Add PBS to the tube to bring the final volume to 50 ml.

Count the cells by using a hemocytometer.

Pellet the cells by centrifugation at 1500 rpm for 5 minutes.

Remove the supernatant, re-suspend with 50 ml of PBS (with Ca2+ and Mg2+) and spin again.

Resuspend the cells to 2×10⁷ per ml and transfer 0.8 ml per electroporation cuvette

Electroporate in a Biorad electroporator at:
Voltage: 200V
Capacitance: 975 μF
Resistant: 200 Ohm
Cuvette 4 mm
Time Constant should be 15-20 millisec

After electroporation, wait for 5 minutes at room temperature and seed ES cells onto 15 cm plates containing half of normal no MEF's and 25 ml regular ES cell medium.

Place plates into the 37° C. CO₂ incubator.

### Selection for Recombinant Clones

### Selection by Addition of Geneticin to Electroporated Cells

1 day after electroporation, change medium of 15 cm selection plates containing electroporated cells and add 150 ug/ml geneticin for B6 cells or up to 250 ug/ml for 129 ES cells

keep changing medium every day. Reduce geneticin concentration gradually to 75 ug/ml after about 8-9 days for B6 cells and 100 ug/ml for 129 m cells after about 6-7 days

### Selecting Colonies

Rinse plates thoroughly to remove the entire medium with PBS (containing Ca2+ and Mg2+).

Add approximately 40 ml of PBS (with Ca2+ and Mg2+) into the plate to start selecting

Set the pipetteman to 10 μl; select colonies with appropriate morphology under the microscope and transfer the colony into a empty 96-well plate.

When the plate is filled split the clones into another 96 well-plate as follows:

Add 45 μl of Trypsin in to each well (from the original plate)

Incubate in the 37° C. incubator for about 10 minutes

Pipette up and down 15 times (approximately) to achieve single cell suspension

Transfer everything into a new 96 well-plate containing 150 ul medium

Grow ES-cell colonies in the incubator for further expansion.

### Expansion of Colonies in 96 Well Plates

Observe the cells everyday under microscope

Change medium daily

### Split the 96 Well Plates

Remove the medium

Wash the plate twice with PBS

Add 45 μl of Trypsin into each well

Place into incubator for 5 minutes

Add 110 μl of medium to stop the digestion

Use the pipette tip to draw up and down 10 times to separate the cells

Transfer 35 μl of the cells (from each well) into 3 new 96 well plates containing 150 ul medium

Keep the remaining cells in the original master plate.

At this point we should have 4 plates (2 will be used for DNA extraction, the other 2 plates will be frozen for subsequent retrieval of targeted clones)

ES Cell Derivation from Blastocysts
1. 3.5 dpc blastocysts are isolated from the uterus following standard methods. Uteri are flushed in ES cell medium that is prewarmed to 37°.
2. Rinse blastocysts through at least two clean drops of ES cell medium and plate onto one well of a 4-well dish (or a 24-well dish, 1 cm diameter). This dish is preplated with STO feeders the day before, 1.5×10⁵ cells per dish. SLN/c STO cells contain Lif and Neo and were obtained from Jeff Mann. ES cell medium supplemented with 50 uM (micromolar) PD98059, a MEK1 inhibitor (Cell Signaling Technologies #9900) is used. Use the supplement during the blast outgrowth process and until you have colonies. Once colonies are growing, you can stop using the MEK1 inhibitor.
3. Monitor the ICM outgrowth closely. It usually takes 2 days for the blastocysts to hatch and attach to the bottom of the dish. Then another 2-3 days for the outgrowth to reach optimal size. Refer to Nagy et al for examples.
4. When the outgrowth is ready, it should be trypsinized into clumps of cells (do not try and get a single cell suspension) and placed onto a new feeder plate with the MEK inhibitor in the medium. Two alternative methods are provided below.

A. If experienced with a mouth pipette, pick up the outgrowth with a pulled glass pipette and wash through 2 drops of PBS and place it into a small drop of trypsin (0.25% trypsin/EDTA (Gibco Cat #25200-072) and placed at 37°. After about 5 minutes the plate is removed from the incubator. A pipette prefilled with ˜200 microliters of complete medium is used to flood the drop of trypsin with medium and then manually dissociate the trypsinated outgrowth.

B. Alternatively, the entire well can be dissociated with trypsin as if it were already an ES cell line.

5. After several more days in culture (P1) colonies might be seen. At this point, the MEK inhibitor can be removed and cells treated like any normal ES cell line.

The foregoing protocols are exemplary only. The skilled person appreciates that they may be varied while still achieving the objectives of the invention.

### Example I

### Improved Growth Conditions for Mouse Embryonic Stem Cells (M ES Cells)

To make mouse ES cells more suitable for high throughput use, improved growth conditions for our existing B6 ES cell lines were established. Multiple growth factors and cytokines, such as LIF, BMP, FGF, TGF and others have been shown to play an important role in self renewal of mouse ES cells and are frequently added to the culture medium either as recombinant proteins or via the addition of serum and/or mouse embryonic fibroblasts (MEF's) to ensure proper maintenance of ES cell germ line competence and growth (8-10). In the present example, we describe studies designed to assess whether ES cells and/or MEF's themselves secrete growth factors or cytokines into the medium that act in autocrine and/or paracrine fashion to promote self renewal and pluripotency of ES cells. It has long been known that doubling times in mouse ES cell lines differ. Other observed differences include capacity of germ line transmission and genetic stability. 129 lines are known to grow more vigorously than B6 mouse ES cell lines, are more likely to go through germ line, and are less feeder cell dependent (6). We wished to determine whether this vigorous growth and reduced dependency on feeder cell layers observed in 129 ES cells was due in part, to secretion of growth factors into the medium that strongly stimulate 129 ES cells via a paracrine/autocrine feedback loop. To test this hypothesis, 129 ES cells (R1 cells) were grown in our normal medium for 24 h.

Normal ES medium

| | | |
|---|---|---|
| DMEM | 400 | ml |
| FCS (ES cell qualified) −20° C. | 80 | ml |
| L-glu 100X (−20° C.) | 5 | ml |
| NEAA 100X (4° C.) | 5 | ml |
| HEPES 1M (4° C.) | 5 | ml |
| Pen/Strep (antibiotic) 100X (4° C.) | 5 | ml |
| LIF (10⁷ u/ml) (4° C.) | 50 | μl |
| beta-Mercaptoethanol 55 mM (4° C.) | 500 | μl |
| (Tissue Culture Grade) | | |

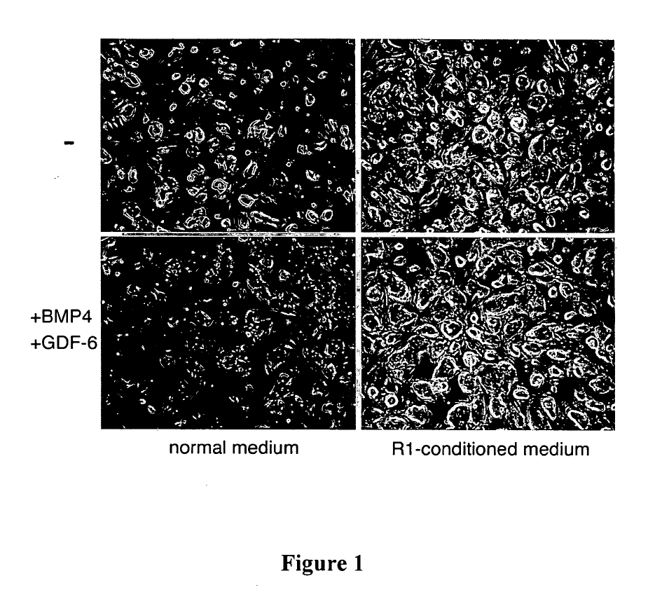

The resulting 129-conditioned medium was then filtered through a standard tissue culture filter. This 129-conditioned medium was added to wells of a 24 well plate containing various amounts of MEF's and B6 or 129 ES cells (see Table 1 below). In addition, several labs have recently reported on the feeder-less growth of 129 Ola (E14Tg2a) ES cells which indicated that feeder independence is possible in the presence of Bone Morphogenetic Protein (BMP4) and/or Growth Differentiation Factor (GDF-6) (1, 3). To test if BMP4 and/or GDF-6 could make B6 cells less dependent on MEF's, these reagents were added to normal or 129-conditioned medium. The conditions and results are summarized in Table 1 and FIG. 1.

TABLE 1
B6 ES cells are strongly stimulated by
129-conditioned medium and BMP4/GDF-6

| | | MEFs seeded per well | | |
|---|---|---|---|---|
| ES cells | Medium | 1 × 10⁵ | 5 × 10⁴ | 0 |
| 129 | normal | +++ | +++ | ++ |
| B6 | normal | ++ | +++ | + flat |
| B6 | Normal + BMP4 + GDF6 | ++ | +++ | + flat |
| B6 | 129-condtioned | ++ | +++ | +++ |
| B6 | 129-condtioned + | +++ | +++ | +++ |
| | BMP4 + GDF6 | | | |

MEFs were seeded at the indicated density into a 24 well plate one day before the B6 ES cells. 2 × 10⁵ B6 or 129 ES cells were added to each well. The wells that received no MEF's prior to addition of ES cells still contained some MEFs carried over from the ES cell plate (see also FIG. 1). B6 ES cells were grown in the presence of either normal ES medium (standard preparation includes 15% FCS and 500 units/ml LIF) or 129-conditioned medium with or without added 20 ng/ml recombinant BMP4 (R&D) and 300 ng/ml recombinant GDF-6 (R&D). 26 h after seeding the ES cells growth characteristics were monitored by estimating the culture time required between passages:
+ = slow/>4 days;
++ = medium/~3 days;
+++ = fast/~2 days;
++++ = very fast/~24 hours;
flat = more than 50% of colonies differentiated into fibroblast-like cells.

Several conclusions can be drawn from these studies. First, while 129 ES cells grow rapidly on MEF's regardless of feeder density, B6 ES cells grow optimally at a narrow feeder density. Using standard ES medium, B6 ES cells both grow slowly and differentiate in the absence of MEFs. In contrast, they grow well at intermediate feeder density, but less so at high MEF density. Second, growth and undifferentiated morphology of B6 ES cells can be improved dramatically by the addition of conditioned medium from 129 ES cell cultures (see Table 1 and FIG. 1), allowing growth rates of B6 ES cells that exceed those of 129 ES cells even if they are seeded onto plates lacking MEFs. It remains to be established if this holds true once B6 ES cells have been cultured for multiple passages without feeder layers. Nevertheless, these results are extremely promising. These data establish the following:

1. that B6 ES cells are not intrinsically less proliferative than 129 ES cells;
2. that B6 ES cells are much less feeder-dependent when grown with 129 conditioned medium; and
3. that 129 ES cells secrete growth factors and/or cytokines into the medium in sufficient quantities to stimulate B6 ES cell growth and inhibit B6 ES cell differentiation.

In summary, the data presented herein indicate that conditions have been identified that substantially increase the rate of proliferation of B6 ES cell lines to a rate that is similar to or exceeds that of 129 lines and make them less dependent on MEF's.

### Feederless Culture of ES Cells and the Effect of Matrigel Or Matrigel Components on the Growth of Mouse ES Cells

Matrigel is known to support growth of human ES cells in the absence of feeder cells although it is not clear how the gel affects maintenance of totipotency over multiple passages (11,12). Matrigel consists of extracellular matrix proteins (collagen, laminin, fibronectin, vitronectin) and small amounts of growth factors (TGF, etc). It is a patented commercially available extract from growth medium derived from a sarcoma culture that produces large amounts of ECM proteins and its exact composition is not published. The main manufacturer is BD Biosciences.

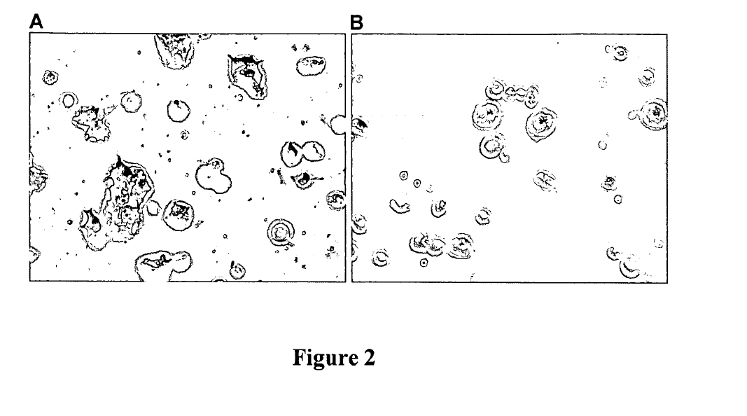

Matrigel was assessed for its capacity to support feederless growth of mouse ES cells in the presence and absence of ES/MEF-derived conditioned medium. The data show that there is indeed a dramatic effect of matrigel on the shape of ES colonies on matrigel versus gelatin in the absence of feeders. See FIG. 2.

### Example 2

### Identification and Characterization of the Growth Factor(s) Present in 129 Conditioned Medium

The improved growth conditions described above will be assessed for their ability to preserve and/or increase totipotency of the mouse ES cells, using several known and newly derived B6 ES cell lines. These lines will be passages between 10 and 20 times and will be karyotyped to directly test germ line transmission rates.

To identify the growth factors that are found predominantly in 129-conditioned medium but not B6-conditioned medium, expression profiling will be employed. The Agilent mouse chip should reveal the mRNA expression of every known mouse gene in ES cells grown in conditioned and normal medium. Using bio informatics we will identify differentially expressed genes that are likely to be excreted into the medium.

The candidate growth factors emerging from this screen will then be added to normal ES medium and their ability to recapitulate the beneficial effects of conditioned medium observed on growth of B6 ES cells will be assessed.

We will also test if the growth factors BMP and GDF-6 as well as the factors emerging from the above screen can substitute for the addition of serum to the growth medium. We will test if the above factors can substitute for feeders. The absence of feeder cells would simplify the use of robotics to grow B6 ES cells in small and large dishes and to pick antibiotic resistant colonies from selection plates. Absence of feeders would also allow much easier manual handling of B6 ES cells, thus allowing their use in high throughput settings.

We will add 129 Sv×129 SvJ (R1)-conditioned medium with or without BMP4 and GDF-6 in the absence of added MEF. We will perform this with our four best normal and albino B6 lines as an initial assessment of their maximal capacity for growth while maintaining undifferentiated colony morphology. We will also test the effect of several other growth factors that are known to be important in self-renewal of mouse ES cells, such as Transforming Growth Factor (TGF) and Fibroblast Growth Factor (FGF) (13-15) on our B6 ES cell lines. It is possible that the R1-conditioned medium we have used successfully in the B6 line contains some of these factors. Table 4 describes the different combinations of conditioned medium and growth factors to be employed.

Serum free medium appears to be sufficient for the feeder-independent growth and self-renewal of the E14Tg2a ES cells if LIF and BMP are present (10). These data raise the possibility of omitting both serum and MEFs for the culture of B6 ES cells, which would lead to significant cost and time savings in a high throughput setting. We will attempt to grow our B6 cell lines serum free/MEF free and in the presence of a variety of growth factors (see Table 4). If successful, we will evaluate for how many passages our B6 cells can be cultivated without losing germ line competence. We anticipate that additional unknown growth factors will have to be added to the medium for MEF-free culture of B6 ES cells to maintain germ line competence over more than 30 passages. Below, we describe our strategy for the identification of such additional growth factors. These novel growth factors will be tested similar to BMP, GDF, FGF and TGFbeta (see Table 2).

TABLE 2
Identification of optimal growth conditions for all B6 ES

A) 129-conditioned medium + growth factors
R1-conditioned medium
R1-conditioned medium + BMP4
R1 conditioned medium + GDF-6
R1-conditioned medium + FGF
R1 conditioned medium + TGFβ
R1-conditioned medium + BMP4 + GDF-6
R1 conditioned medium + BMP4 + GDF-6 + FGF
R1-conditioned medium + BMP4 + GDF-6 + TGFβ
R1 conditioned medium + BMP4 + GDF-6 + TGFβ + FGF
B) Serum free medium + growth factors
Serum free medium
Serum free medium + BMP4
Serum free medium + GDF-6
Serum free medium + FGF
Serum free medium + TGFβ
Serum free medium + BMP4 + GDF-6
Serum free medium + BMP4 + GDF-6 + FGF
Serum free medium + BMP4 + GDF-6 + TGFβ
Serum free medium + BMP4 + GDF-6 + TGFβ + FGF

Conditions will be tested by inoculation of a logarithmically growing B6 cultures into a 24-well plate (gelatin-coated, no MEF's) at 2×10⁵ B6 ES cells per well. Growth will be monitored every day for 3 consecutive days and documented by digital phase contrast microscopy (see FIG. 2). BMP4 and GDF-6 will be added at 20 ng/ml and 300 ng/ml per well respectively. FGF and TGFβ will be used as described (13-15).

Based on the initial results we will change the types of growth factors to be added as well as the precise combinations to be used. Because ES cell lines vary widely regarding their growth characteristics, even if they were isolated from the same mouse strain, we anticipate that different B6 lines will have different preferences for growth factors and will adapt our testing strategy accordingly. Any growth factors emerging from our expression profiling approach described above will be tested in an analogous way, either alone or in various combinations with existing growth factors. Again, we anticipate that different cell lines will exhibit different preferences for growth factors and we will adjust our testing protocol accordingly.

After we have established the optimal growth conditions from several rounds of testing, we will evaluate the two best B6 lines to confirm that growth rates and morphology are stable over many passages.

First, we will grow the two B6 lines for several consecutive passages under the new conditions. This will reveal if these conditions work even in the complete absence of feeders. If this is the case we will proceed to the second test below. If not we will employ the well-characterized STO cell line transfected with the various growth factors we have identified. Stably transfected STO cells are easy to derive, can be cultured indefinitely and thus should save money and time in a high throughput setting.

Second, we will grow these two lines under the final optimal conditions for fifteen and thirty passages, respectively, and subject them to our newly developed gene profiling test for germ line competence. In addition we will measure directly by immunofluorescence and western blotting the levels of Oct4, Nanog, Foxd3, SSEA-1 and other proteins whose levels are indicative of proper self renewal.

Expression profiling will be performed on 129 (R1) and B6 (Chemicon) embryonic stems cells as well as newly derived B6 embryonic stem cells grown under identical conditions (same batch of MEF, same medium). We will prepare total RNA from 10 cm dishes grown to 50% confluency, with 5 replicates for each ES-cell line. We will employ Agilent's whole mouse genome oligo microarray for the expression profiling. This platform consists of 41,534 60-mer oligonucleotide probes representing over 41,000 mouse genes and transcripts. All the equipment and software programs for the hybridization and analysis of these slides including the Agilent G2505B scanner, which reduces the cost of these experiments compared to a core facility are available to us for this purpose.

Total RNA will be extracted using the RNeasy method (Qiagen). Each RNA sample will be subjected to Bioanalyzer analysis, which allows for the simultaneous determination of RNA quantity and quality. RNA samples with a 285 to 18S ratio of less than 2.0 will be excluded from further analysis and replaced with better samples. Indirect labeling of cDNA with fluorescent dyes is the most reproducible and quantitative method currently available and thus will be employed in these studies (16). This labeling method requires only 10 μg of total RNA, which will be easily isolated from a 10 cm culture dish, thus eliminating the need for amplification of the RNA samples.

ES cell RNA samples (10 μg) will be reverse-transcribed to incorporate amino-allyl dUTP as previously described (17). The cDNAs will be coupled to either CyS or Cy3 fluorescent dyes (GE) using a modified indirect labeling protocol (17). Coupled samples will be combined, purified with Qiaquick PCR Purification Kit [Qiagen], and eluted in 18.5 μl sterile de-ionized water. 2.5 μl oligo(dT)21 blocker (0.5 mg/ml) and 2.5 μl human Cot1 DNA (1 mg/ml) [Invitrogen] will be added to the cDNA and incubated at 95° C. for 5 min. An equal volume of pre-warmed (42° C.) 2× Hybridization Buffer (50% Formamide, 10×SSC, 0.2% SDS) will be added and the sample transferred to a pre-hybridized glass array, covered with a coverslip (22×60 mm) and incubated overnight in a Corning hybridization chamber at 42° C. The coverslip will be removed from the labeled array in 2×SSC, 0.1% SDS. The arrays will then be washed two times for 5 min each with agitation: once at 40° C. in 0.2×SSC, 0.1% SDS and once in 0.2×SSC at room temperature and then dried by centrifugation in a slide rack for 3 min at 1000 rpm.

Scanning and Image analysis. All slides will be scanned immediately following hybridization and washing using our Agilent G2505B scanner. Image analysis will be performed with GenepixPro 5.0 software. Signal and background intensities will be determined by the median pixel values.

### Microarray Data Analysis.

A streamlined procedure for the analysis and statistical evaluation of microarrays which allows for rapid data acquisition is required for the large number of samples to be analyzed. After quantification of median spot intensity using GenePix, images are visually inspected and hybridizations repeated for low quality slides. The data are then normalized using the statistical software package developed by T. Speed and colleagues (18) including Loess curve fitting, print tip and scaled normalization algorithms. The normalized data are thresholded using the blanks (yeast intergenic sequences) included on the array. Finally, the ratio of the relative expression levels (B6 to 129 ES cells) is calculated and the data ranked by the statistical significance and the fold changes in expression levels. The final data table contains the gene name, functional annotation, GenBank Accession, normalized intensities, background intensities, median, mean and standard deviation. In addition, all data will be entered into the MIAME-compliant relational database RAD (RNA abundance database; (19) through a web-based user interface and ported to ArrayExpess. We will utilize two statistical analysis programs, PaGE and SAM (20, 21), to identify genes that are differentially expressed between 129 and B6 ES lines. In a parallel approach, unsupervised clustering (hierarchical, k-means, SOM) will be used to identify genes expressed with similar patterns in the ES cell populations to be compared. Pathway analysis with the Ingenuity Pathway Analysis Program will be employed to identify gene networks, signaling pathways, growth factors and cytokines that are differentially expressed between 129 and B6 ES-cells.

The growth factors and/or cytokines identified above will be tested for their activity towards B6 ES-cells growth as described in Example I. If no commercial source for the factor exists, we will pursue multiple avenues to effect expression of the factor of interest. First, we will attempt to express and purify the recombinant protein itself. Second, we will engineer STO feeder cells to stably over-express the growth factor/cytokine. Third, we will replace the gene and its surrounding sequence containing its cis-regulatory elements in the B6 cells with the same gene from 129 ES cells via homologous recombination.

We will also include a proteomics approach. Given that normal growth medium contains 15% serum, we would perform short-term culture with serum free medium to obtain the proteins secreted by either 129 or B6 ES-cells and subject them to comparative proteomics analysis. The University of Pennsylvania has a state-of-the-art proteomics core (See the world wide web at .med.upenn.edu/ccp/cancer_pharm_bio.shtml) for performance of these analyses.

### Example 3

### Bioassay to Functionally Identify Growth Factors Present in Conditioned Media

Our goal was not to analyze all factors that are ‘in general’ beneficial for B6 ES cell growth (with or without feeder cells) but rather to identify the subset of these factors that allow survival of B6 ES cells in the absence of MEF feeder layers. The main advantage of this assay is its speed: Factor(s) can be identified in two weeks versus about 6 months for assessing the quality of the cells for germ line transmission (The germline transmission assay involves injection of ES cells into mouse blastocysts and breeding of the resulting chimeric mice). The B6 survival factors, as identified herein, when added to the normal growth medium will be beneficial in long term culture of B6 ES cells.

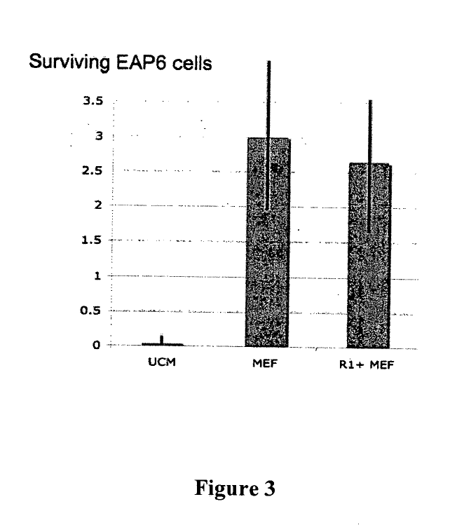

Our research has revealed that certain factors are common to both MEF-only conditioned medium and MEF+129 conditioned medium (see FIG. 3). It appears that those factors that allow survival of B6 ES cells in the two week bio assay described herein are present in both MEF conditioned medium and in MEF+129 conditioned medium.

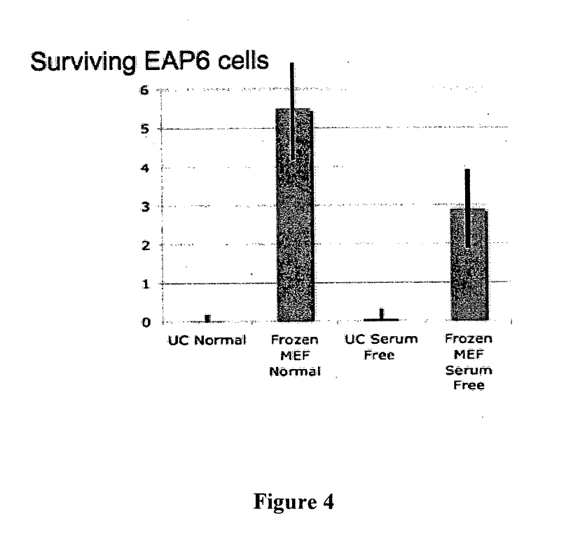

We have also determined that the growth factors present in conditioned media can be frozen without loosing biological activity (see FIG. 4). Thus, the media will be subjected to biochemical fractionation, and purification to further identify and characterize the biologically active factors that enhance B6 cell survival.

Our data also show that the growth factors are excreted into serum free medium during a 24 h period (see FIG. 4) facilitating straight forward biochemical purification and characterization.

The biological assay described herein facilitates functional identification of B6 specific growth factors necessary for survival of B6 ES cells in the absence of MEF feeder layers. We also show that the growth factors can be frozen without significant loss of activity and that the factors are secreted by MEF into serum free medium.

The media will subjected to the following purification procedures:

1. Reversible binding of factor(s) to basic and acidic ion exchange resin will be determined to identify the correct resin (e.g. MonoQ or Mono S) and concentrate factors ˜100 fold;
2. Factors will be eluted by FPLC using a linear salt gradient thereby enhancing purification up to 50 fold;
3. Factors will optionally be further purified to homogeneity by size exclusion chromatography; and
4. Active factor(s) will be identified by Mass Spectrometry.

Thus, the aforementioned assay provides enables the identification, biochemical characterization and purification of those growth factors that enhance growth, viability and frequency of germ line transmission of mouse ES cells.

### REFERENCES

1. Austin, C. P., Battey, J. F., Bradley, A., Bucan, M., Capecchi, M., Collins, F. S., Dove, W. F., Duyk, G., Dymecki, S., Eppig, J. T., et al. 2004. The knockout mouse project. Nat Genet 36:921-924.
2. Simpson, E. M., Linder, C. C., Sargent, E. E., Davisson, M. T., Mobraaten, L. E., and Sharp, J. J. 1997. Genetic variation among 129 substrains and its importance for targeted mutagenesis in mice. Nat Genet 16:19-27.
3. Linder, C. C. 2001. The influence of genetic background on spontaneous and genetically engineered mouse models of complex diseases. Lab Anim (NY) 30:34-39.
4. Crawley, J. N., Belknap, J. K., Collins, A., Crabbe, J. C., Frankel, W., Henderson, N., Hitzemann, R. J., Maxson, S. C., Miner, L. L., Silva, A. J., et al. 1997. Behavioral phenotypes of inbred mouse strains: implications and recommendations for molecular studies. Psychopharmacology (Berl) 132:107-124.
5. Puglisi-Allegra, S., and Cabib, S. 1997. Psychopharmacology of dopamine: the contribution of comparative studies in inbred strains of mice. Prog Neurobiol 51:637-661.
6. Cheng, J., Dutra, A., Takesono, A., Garrett-Beal, L., and Schwartzberg, P. L. 2004. Improved generation of C57BL/6J mouse embryonic stem cells in a defined serum-free media. Genesis 39:100-104.
7. te Riele, H., Maandag, E. R., and Berns, A. 1992. Highly efficient gene targeting in embryonic stem cells through homologous recombination with isogenic DNA constructs. Proc Natl Acad Sci USA 89:5128-5132.
8. Qi, X., Li, T. G., Hao, J., Hu, J., Wang, J., Simmons, H., Miura, S., Mishina, Y., and Zhao, G. Q. 2004. BMP4 supports self-renewal of embryonic stem cells by inhibiting mitogen-activated protein kinase pathways. Proc Natl Acad Sci USA 101:6027-6032.
9. Varga, A. C., and Wrana, J. L. 2005. The disparate role of BMP in stem cell biology. Oncogene 24:5713-5721.
10. Ying, Q. L., Nichols, J., Chambers, I., and Smith, A. 2003. BMP induction of Id proteins suppresses differentiation and sustains embryonic stem cell self-renewal in collaboration with STAT3. Cell 115:281-292.
11 Tenneille E Ludwig, Veit Bergendahl, Mark E Levenstein, Junying Yu, Mitchell D Probasco & James A Thomson 2006 Feeder-independent culture of human embryonic stem cells Nature Methods 3:637-646
12 Ludwig T E, Levenstein M E, Jones J M, Berggren W T, Mitchen E R, Frane J L, Crandall L J, Daigh C A, Conard K R, Piekarczyk M S, Llanas R A, Thomson J A. 2006 Derivation of human embryonic stem cells in defined conditions. Nature Biotechnology 24:185-187
13. Chambers, I. 2004. The molecular basis of pluripotency in mouse embryonic stem cells. Cloning Stem Cells 6:386-391.
14. O'Shea, K. S. 2004. Self-renewal vs. differentiation of mouse embryonic stem cells. Biol Reprod 71:1755-1765.
15. Rao, M. 2004. Conserved and divergent paths that regulate self-renewal in mouse and human embryonic stem cells. Dev Biol 275:269-286.
16. Manduchi, E., Scearce, L. M., Brestelli, J. E., Grant, G. R., Kaestner, K. H., and Stoeckert, C. J. 2002. Comparison of different labeling methods for 2-channel high-density microarray experiments. Physiol. Genomics in revision.
17. Kaestner, K. H., Lee, C. S., Scearce, L. M., Brestelli, J. E., Arsenlis, A., Le, P. P., Lantz, K. A., Crabtree, J., Pizarro, A., Mazzarelli, J., et al. 2003. Transcriptional Program of the Endocrine Pancreas in Mice and Humans. Diabetes 52:1604-1610.
18. Yang, Y. H., Dudoit, S., Luu, P., Lin, D. M., Peng, V., Ngai, J., and Speed, T. P. 2002. Normalization for cDNA microarray data: a robust composite method addressing single and multiple slide systematic variation. Nucleic Acids Res 30:e15.
19. Manduchi, E., Grant, G. R., He, H., Liu, J., Mailman, M. D., Pizarro, A. D., Whetzel, P. L., and Stoeckert, C. J., Jr. 2004. RAD and the RAD Study-Annotator: an approach to collection, organization and exchange of all relevant information for high-throughput gene expression studies. Bioinformatics 20:452-459.
20. Manduchi, E., Grant, G. R., McKenzie, S. E., Overton, G. C., Surrey, S., and Stoeckert, C. J., Jr. 2000. Generation of patterns from gene expression data by assigning confidence to differentially expressed genes. Bioinformatics 16:685-698.
21. Tusher, V. G., Tibshirani, R., and Chu, G. 2001. Significance analysis of microarrays applied to the ionizing radiation response. Proc Natl Acad Sci USA 98:5116-5121.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for enhancing the growth of mouse embryonic stem cells on non-confluent feeder layers comprising supplementing the culture medium with conditioned ES/MEF culture medium.

2. The method of claim 1 wherein said embryonic stem cells are B6 cells and said ES/MEF culture medium is obtained from cultured mouse 129 ES cells.

3. The method of claim 1 wherein the supplemented growth medium comprises about three quarters of normal growth medium and about one quarter of ES/MEF-conditioned growth medium.

4. The method of claim 3 wherein the supplemented growth medium further comprises BMP 3/4 and optionally GDF6

5. The method of claim 3, wherein the supplemented growth medium comprises at least one growth factor selected from the group consisting of BMP3/4, GDF6, bFGF and TGF beta.

6. A method for culturing mouse ES cells in the absence of feeder layers comprising seeding the cells onto a matrix and supplementing the growth medium with conditioned ES/MEF culture medium.

7. The method of claim 6, wherein said matrix is matrigel.

8. The method of claim 7 wherein matrigel is diluted between about 2-4 fold in DMEM media

9. The method of claim 7 wherein matrigel is diluted more than four fold in DMEM media.

10. The method of claim 6, wherein said matrix is formed by laminin.

11. The method of claim 6, wherein said matrix is formed by fibronectin.

12. The method of claim 6, wherein said matrix is formed by a combination of laminin and fibronectin.

13. The method of claim 12, wherein said combination further comprises either collagen, vitronectin or both.

14. A growth factor isolated from the conditioned medium of claim 1.

15. A cytokine isolated from the conditioned medium of claim 1.

16. A method for identifying factors which promote B6 cell survival in cultures lacking feeder cells, comprising:
a) providing cultures of B6 cells, said cultures being incubated in the presence of
i) unconditioned (normal) medium (UCM);
ii) MEF conditioned medium (MEF); or
iii) 129+MEF conditioned medium;
b) passaging said cells at least five times in the absence of MEF;
c) determining cell viability and proliferation in i), ii) and iii); and
d) purifying at least one factor from the medium of ii) thereby isolating said factor(s) which increase B6 cell survival.

17. A growth factor identified by the method of claim 16.

18. The method of claim 16, further comprising isolating and purifying at least one growth factor from the media of iii).

19. A growth factor identified by the method of claim 18.

20. The method of claim 16, wherein said media are frozen prior to purification of said at least one factor.
